(19) United States
(12) Patent Application Publication
(10) Pub. No.: US 2008/0154092 A1
(43) Pub. Date: Jun. 26, 2008

(54) Capsule Type Endoscope with Embedded Memory

(75) Inventors: Dae Sung Lee, Gyeonggi-do (KR); Nam Kyu Cho, Gyeonggi-do (KR); Kyu Sik Shin, Seoul (KR); Hak In Hwang, Gyeonggi-do (KR)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: KOREA ELECTRONICS TECHNOLOGY INSTITUTE, Gyeonggi-do (KR)

(21) Appl. No.: 11/964,124

(22) Filed: Dec. 26, 2007

(30) Foreign Application Priority Data
Dec. 26, 2006 (KR) 10-2006-0134175

(51) Int. Cl. A61B 1/04

(52) U.S. Cl. 600/109

(57) ABSTRACT

The present invention relates to a capsule type endoscope with embedded memory. More specifically, the present invention relates to a capsule type endoscope with embedded memory, which is equipped with flash memory and does not need a transmitter for wireless communication or a receiver, to reduce power consumption of the capsule type endoscope and improve convenience in use.

### BACKGROUND OF THE INVENTION

1. Field of the Invention

The present invention relates to a capsule type endoscope with embedded memory. More specifically, the present invention relates to a capsule type endoscope with embedded memory, which is equipped with flash memory and does not need a transmitter for wireless communication or a receiver, to reduce power consumption of the capsule type endoscope and improve convenience in use.

2. Background of the Related Art

Any discussion of the prior art throughout the specification should in no way be considered as an admission that such prior art is widely known or forms part of common general knowledge in this field.

FIG. 1 illustrates a construction of a conventional capsule type endoscope. Referring to the FIG. 1, the conventional capsule type endoscope comprises a subminiature camera 130, a light-emitting diode (LED) illuminator 140, a controller, an antenna 100, a wireless transmitter 110, various sensors, and a battery 120. Such capsule type endoscope is used for taking pictures or images or collecting information about a temperature, a pressure, etc., inside a digestive organ.

The collected information is transmitted to and then stored in an information-receiver or an information-recorder, etc., through the wireless transmitter 110 or the antenna 100. At this time, the information-receiver or the information-recorder, etc. are carried outside a subject's body. After that, the stored data are displayed on a display and analyzed through an analysis device.

When the acquired images or data are transmitted to the information-receiver carried outside the body, the conventional capsule type endoscope consumes a lot of power for transmitting the images or data in a wireless manner. This makes it difficult to take images of the organs from the small intestine through the large intestine.

Accordingly, as one of the ways to minimize consumption of power, a level of power output of the wireless transmitter 110 is required to be lowered. And a distance between the capsule type endoscope and the information-receiver outside a subject's body must be minimized to lower a level of power output of the wireless transmitter.

In this regard, an information-collecting apparatus using the conventional capsule type endoscope has the disadvantage that a subject must put on an information-receiver on his or her abdominal region so that the information-receiver can receive information from the conventional capsule type endoscope which he or she has swallowed.

### SUMMARY OF THE INVENTION

Accordingly, the present invention is directed to a capsule type endoscope with embedded memory that substantially obviates one or more problems due to limitations and disadvantages of the related art.

An object of the present invention is to provide a capsule type endoscope with embedded memory, which can collect information about an interior of a body without an antenna or a transmitter for wireless transmission.

Another object of the present invention is to provide a capsule type endoscope with embedded memory, which can minimize consumption of power.

Still another object of the present invention is to provide a capsule type endoscope with embedded memory, which does not use an information-receiver, thus improving convenience in use.

To accomplish the above objects, according to one aspect of the present invention, there is provided a capsule type endoscope with embedded memory, comprising at least one illuminating section for illuminating an interior spot or area of a body; an image-sensing section for acquiring image information of the interior spot or area which the illuminating section illuminates; a signal-processing section for controlling the image-sensing section and compressing data corresponding to the acquired image information; a storing section for accumulatively storing the compressed data until the capsule type endoscope is discharged from the body; a controlling section for controlling the signal-processing section and the storing section; and a power-supplying section for supplying power to the capsule type endoscope.

To accomplish the above objects, according to one aspect of the present invention, there is also provided a capsule type endoscope with embedded memory, comprising: an environmental information-sensing section equipped with at least one of a pressure sensor, a temperature sensor, a vibration sensor and a pH sensor, for sensing environmental information in a body; a signal-processing section for controlling the environmental information-sensing section and collecting data corresponding to the environmental information by processing a signal sensed by the environmental information-sensing section; a storing section for accumulatively storing the collected data until the capsule type endoscope is discharged from the body; a controlling section for controlling the signal-processing section and the storing section; and a power-supplying section for supplying power to the capsule type endoscope.

It is to be understood that both the foregoing general description and the following detailed description of the present invention are exemplary and explanatory and are intended to provide further explanation of the invention as claimed.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings;

FIG. 1 illustrates a construction of a conventional capsule type endoscope;

FIG. 2 illustrates a construction of a capsule type endoscope with embedded memory according to an embodiment of the present invention;

FIG. 3 is a block diagram which shows a capsule type endoscope with embedded memory according to an embodiment of the present invention; and

FIG. 4 is a block diagram which shows a capsule type endoscope with embedded memory according to another embodiment of the present invention.

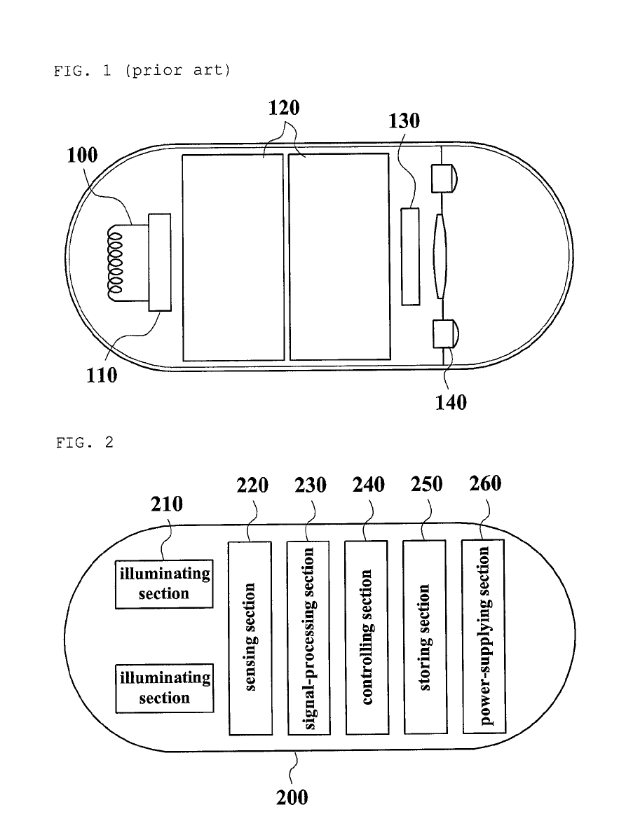

### DETAILED DESCRIPTION OF THE PREFERRED EMBODIMENT

The present invention will now be described more fully with reference to the accompanying drawings, in which exemplary embodiments of the invention are shown. The invention may, however, be embodied in many different forms and should not be construed as being limited to the embodiments set force herein, rather, these embodiments are provided so that this disclosure will be thorough and complete, and will fully convey the concept of the invention to those skilled in the art.

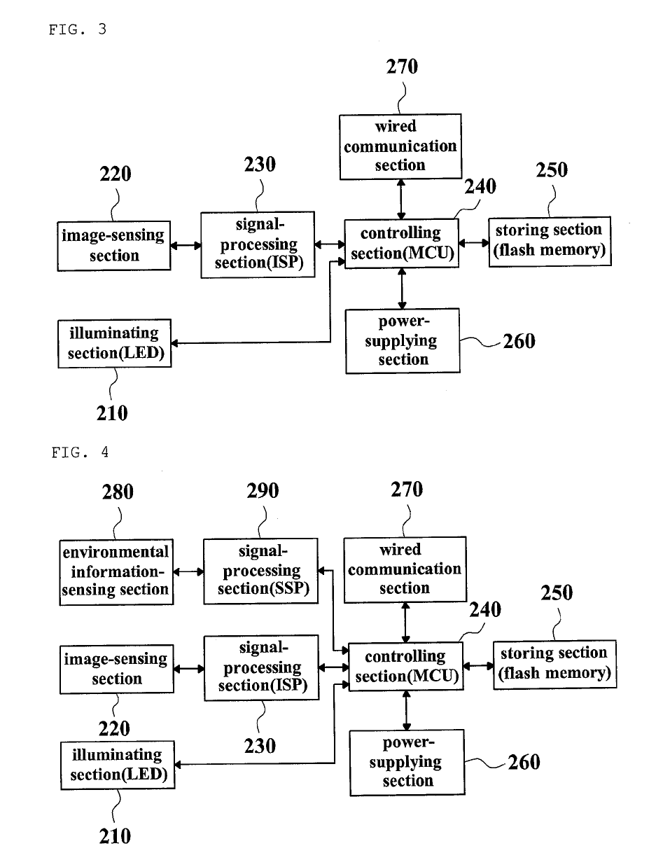

FIG. 2 illustrates a construction of a capsule type endoscope according to an embodiment of the present invention; and FIG. 3 is a block diagram which shows a capsule type endoscope according to an embodiment of the present invention. Referring to FIG. 2 and FIG. 3. A capsule type endoscope 200 with embedded memory according to an embodiment of the present invention comprises an illuminating section 210, an image-sensing section 220, a signal-processing section 230, a storing section 250, a controlling section 240 and power-supplying section 260.

The illuminating section 210 illuminates an interior spot or area of a body which the capsule type endoscope 200 with embedded memory is to take an image of while passing through the body. The illuminating section 210 is preferably equipped with at least one light-emitting diode (LED).

The image-sensing section 220 captures an image of the spot or area which the illuminating section 210 illuminates inside a body. It is preferred that the image-sensing section 220 is equipped with at least one charge-coupled device (CCD) or complementary metal-oxide semiconductor (CMOS) image sensor.

The image-sensing section 220 is controlled by the signal-processing section 230. The signal-processing section 230 compresses and outputs the data which correspond to the image information acquired and outputted by the image-sensing section. The image-sensing section 220 acquires the image information in the format of bitmap, and the signal-processing section preferably compresses the bitmap file to a JPEG file. In addition, it is preferred that the signal-processing section 230 is an image signal processor (ISP).

Meanwhile, for the conventional capsule type endoscopes as well as the capsule type endoscopes according to the present invention, it physiologically takes about 24 hours to pass through a body to be discharged therefrom. The conventional capsule type endoscope transmits interior image information each time it acquires the interior image information. On the contrary, the capsule type endoscope according to the present invention acquires and collects interior image information of a body for about 24 hours till it being discharged from the body, and, the interior image information is read after the capsule type endoscope comes out of the body.

Accordingly, the capsule type endoscope in accordance with the present invention needs memory big enough to store the interior image information acquired for about 24 hours. In this regard, the capsule type endoscope according to the present invention is made to include flash memory of a high capacity in the storing section 250.

In addition, the storing section 250 accumulatively stores the image information data which have been compressed in the signal-processing section 230, until the capsule type endoscope is discharged from a body. More image information can be stored in a storing section when compressed than when not compressed. Accordingly, with information stored without compression, it is difficult to make a right diagnosis in a medical examination.

For example, in the case that image information is acquired by the image-sensing section 220 of which resolution is 320×320, and stored in the 24 bit image file format of bitmap, a screen comes to have a data capacity of 302 Kbytes. In other word, in the case of acquiring the image information data of 2 frames per second for 24 hours in the format of 24 bit bitmap, memory for storing a total of 52.185 GBytes is needed.

However, in the case that the bitmap is compressed to a JPEG, the data capacity of 320×320 JPEG image is about 30 Kbytes at the compression ratio of 10. In this case, if interior image information is stored for 24 hours, the data capacity of the stored image information becomes 5.1 GBytes (30 Kbytes×2 frames/sec×24 hrs×3600 secs/hr=5.1 GBytes).

Accordingly, the capsule type endoscope 200 is equipped with a flash memory chip with a capacity of about 5 Gbytes in its storing section 250, so as to collect interior image information till being discharged from a body. This enables a subject to be examined without separate sections for receiving and sending image information, in his or her daily life.

A controlling section (micro controller unit, MCU) 240 controls the signal-processing section 230 to collect interior image information through the image-sensing section. And the controlling section gets the image information to be stored in flash memory of the storing section 250, as the image information is compressed in the signal-processing section 230 and outputted therefrom.

After the capsule type endoscope collects the interior image information in this way and is discharged from a body, the collected image information is read.

The flash memory of the storing section 250 is separated from the other parts of the capsule type endoscope. And then, the interior image information stored in the flash memory can be read using a reading terminal for reading flash memory.

In addition, the image information stored in the flash memory can be read through a wired communication section 270, which is equipped in the capsule type endoscope 200. The wired communication section 270 comprises a connector for supporting wired communication. The connector, which is disposed on an external side of the capsule type endoscope 200, can be connected to the reading terminal by a cable after the capsule type endoscope being discharged from a body, so that the image information stored in the flash memory can be read to be analyzed.

Meanwhile, a terminal for displaying the read image information on a display can be combined with the reading terminal or provided separately.

FIG. 4 is a block diagram which shows a capsule type endoscope according to another embodiment of the present invention. Referring to FIG. 4, the capsule type endoscope comprises an environmental information-sensing section 280 in addition to the sections of the capsule type endoscope in accordance with the embodiment in FIG. 2 and FIG. 3.

The environmental information-sensing section 280 senses environmental information of the internal organs. To do this, the environmental information-sensing section 280 comprises at least one of a pressure sensor, a temperature sensor, a vibration sensor, and a pH sensor.

From the information about pressures, temperatures, vibration rates, or pHs acquired through the environmental information-sensing section 280, it is possible to grasp what organ the capsule type endoscope has been located in. This is because each organ has a characteristic temperature, pressure and pH, and so on. Accordingly, the environmental information acquired by the environmental information-sensing section 280 can be compared with the characteristic information of the organs, so as to identify the organs.

In other word, the time when the image-sensing section 220 acquires image information is compared with the time when the environmental information-sensing section 280 acquires environmental information. In this way, image information for each organ can be collected more precisely.

In addition, the environmental information-sensing section 280 is controlled by the signal-processing section 290. The controlling section 240 controls to store the acquired environmental information in the storing section 250. After the capsule type endoscope 200 is discharged from a body, the stored environmental information can be downloaded to the reading terminal through the wired communication section 270, which is disposed on an external side of the capsule type endoscope. At this time, the signal-processing section 290 is a sensor signal processor (SSP), and it processes an environmental information signal sensed through the environmental information-sensing section 280 to acquire data corresponding to the environmental information.

The capsule type endoscopes according to the present invention which are shown in FIGS. 2 to 4 are equipped with a power-supplying section 260. The power-supplying section provides power for the capsule type endoscope to be driven. The power-supplying section 260 can be connected with the controlling section 240 to provide power to each of the components through the controlling section. Or, the power-supplying section can be directly connected with each of the components to provide them with power.

The foregoing embodiments are merely exemplary and are not to be construed as limiting the present invention. The present teachings can be readily applied to other types of apparatuses. The description of the present invention is intended to be illustrative, and not to limit the scope of the claims. Many alternatives, modifications, and variations will be apparent to those skilled in the art.

The capsule type endoscope with embedded memory in accordance with the present invention has the advantage that it does not need an antenna or an information-transmitting section and it can reduce power consumption, by using embedded flash memory.

In addition, the capsule type endoscope with embedded memory in accordance with the present invention has the advantage that it can improve convenience in use, since it uses embedded flash memory, without a need for a separate information-receiver.

What is claimed is:

1. A capsule type endoscope with embedded memory, comprising:
at least one illuminating section for illuminating an interior spot or area of a body;
an image-sensing section for acquiring image information of the interior spot or area which the illuminator illuminates;
a signal-processing section for controlling the image-sensing section and compressing data corresponding to the acquired image information;
a storing section for accumulatively storing the compressed data until the capsule type endoscope is discharged from the body;
a controlling section for controlling the signal-processing section and the storing section; and
a power-supplying section for supplying power to the capsule type endoscope.

2. The capsule type endoscope with embedded memory of claim 1, wherein the storing section comprises flash memory.

3. The capsule type endoscope with embedded memory of claim 2, further comprising:
an environmental information-sensing section equipped with at least one of a pressure sensor, a temperature sensor, a vibration sensor and a pH sensor, for collecting environmental information in the body.

4. The capsule type endoscope with embedded memory of claim 3, further comprising:
a wired communication section for enabling the capsule type endoscope to communicate with an exterior terminal in a wired manner, wherein the exterior terminal reads the data of the storing section.

5. A capsule type endoscope with embedded memory, comprising:
an environmental information-sensing section equipped with at least one of a pressure sensor, a temperature sensor, a vibration sensor and a pH sensor, for sensing environmental information in a body;
a signal-processing section for controlling the environmental information-sensing section and collecting data corresponding to the environmental information by processing a signal sensed by the environmental information-sensing section;
a storing section for accumulatively storing the collected data until the capsule type endoscope is discharged from the body;
a controlling section for controlling the signal-processing section and the storing section; and
a power-supplying section for supplying power to the capsule type endoscope.

6. The capsule type endoscope with embedded memory of claim 5, wherein the storing section comprises flash memory.

7. The capsule type endoscope with embedded memory of claim 6, further comprising:
a wired communication section for enabling the capsule type endoscope to communicate with an exterior terminal in a wired manner, wherein the exterior terminal reads the data of the storing section.
